(19) United States
(12) Patent Application Publication
(10) Pub. No.: US 2018/0030417 A1
(43) Pub. Date: Feb. 1, 2018

(54) METHOD OF ALTERING EXPRESSION OF ALTERNATIVE VIRAL GLYCOPROTEIN COMPLEXES

(71) Applicants: Jeremy Phillip KAMIL, Shreveport, LA (US); William J. BRITT, Birmingham, AL (US); Gang LI, Shreveport, LA (US); Lindsey M. HUTT-FLETCHER, Shreveport, LA (US)

(72) Inventors: Jeremy Phillip KAMIL, Shreveport, LA (US); William J. BRITT, Birmingham, AL (US); Gang LI, Shreveport, LA (US); Lindsey M. HUTT-FLETCHER, Shreveport, LA (US)

(73) Assignee: Board of Supervisors of Louisiana State University and Agricultural and Mechanical College, Baton Rouge, LA (US)

(21) Appl. No.: 15/551,087

(22) PCT Filed: Feb. 16, 2016
(86) PCT No.: PCT/US16/18030

§ 371 Date: Aug. 15, 2017

Related U.S. Application Data
(60) Provisional application No. 62/116,629, filed on Feb. 16, 2015.

(51) Int. Cl. C12N 7/00; A61K 39/245; C07K 14/005

(52) U.S. Cl. CPC C12N 7/00; C07K 14/005; A61K 39/245; C12N 2710/16121; C12N 2710/16134; C12N 2710/16151; A61K 2039/525

(57) ABSTRACT

A method of preparing a vaccine for immunization against a herpes virus comprising the steps of one of deleting, substituting, or modifying a UL148 gene and interfering with or modifying an expression of the UL148 gene. Wherefore, it is an object of the present invention to overcome the above mentioned shortcomings and drawbacks associated with the prior art The inventors observed that less extensively passaged HCMV strains that retain expression of gH/gL/UL128-131 can efficiently infect epithelial and endothelial cells.

### CROSS REFERENCE TO RELATED APPLICATIONS

This application claims priority to U.S. Provisional Patent Application No. 62/116,629 filed Feb. 16, 2015, the contents of which are incorporated herein by reference in its entirety, including the contents of cited references within. To the extent that there is any conflict between the incorporated material and the present application, the present application will control.

### STATEMENT REGARDING FEDERALLY SPONSORED RESEARCH OR DEVELOPMENT

The work described below was supported by Grant Nos. P20GM103433 and P30GM110703, which were awarded by the National Institutes of Health. The Government has certain rights in the invention.

### FIELD OF THE INVENTION

The present invention relates to herpes virus tropism and specifically human cytomegalovirus (HCMV) regulation of viral glycoprotein complexes comprised of dimers of glycoprotein H (gH) and glycoprotein L (gL), (gH/gL) in complex with alternative accessory proteins, glycoprotein O (gO), or UL128, UL130 and UL131.

### BACKGROUND OF THE INVENTION

HCMV causes life threatening opportunistic infections in individuals with compromised immune system function, as is seen in stem cell or solid organ transplantation, HIV-AIDS, and cancer patients undergoing intensive chemotherapy. Furthermore, in the immune-naïve fetus, HCMV is the leading cause of infectious disease-related birth defects resulting in mild to severe hearing loss and cognitive impairment. Congenital HCMV infections outnumber the cases of Downs Syndrome, fetal alcohol syndrome and Spina Bifida in the United States. For these reasons, HCMV has been named the highest priority for vaccine development by the National Vaccine Advisory Committee.

The lipid bilayer membranes of living cells pose an existential challenge to viruses. In non-enveloped viruses, capsid proteins directly mediate entry into cells. In enveloped viruses, viral glycoproteins execute a highly regulated fusion event between virion and cellular membranes, thereby delivering the viral genome and other contents of the virion into the host cell. Antibody responses that block entry are considered neutralizing, and represent an important host defense against viral pathogens.

In many enveloped viruses, one or two viral glycoproteins suffice to carry out binding, attachment and membrane fusion events that mediate entry. In herpesviruses, however, at least four envelope glycoproteins are typically involved. The core machinery for herpesvirus entry is comprised of three highly conserved viral glycoproteins, glycoprotein B (gB), glycoprotein H (gH) and glycoprotein L (gL), along with one or more accessory glycoproteins necessary for binding to cell surface receptors. gB is thought to be the proximal mediator of membrane fusion, while gH and gL form a disulfide-linked complex, termed gH/gL, which has been found to regulate the fusogenic activity of gB. In a number of beta- and gamma-herpesviruses, including the human pathogens human cytomegalovirus (HCMV), human herpesvirus-6 (HHV-6), and Epstein Barr virus (EBV), two different gH/gL complexes are found on the virion envelope, and are necessary for the viruses to enter the full range of cell types they infect in vivo.

In HCMV, a gH/gL complex with glycoprotein O (gO), gH/gL/gO, suffices for entry into fibroblasts, a cell type in which fusion events at the plasma membrane initiate infection. Infection of several other types of cells, including monocytes, dendritic cells, endothelial cells and epithelial cells, requires a pentameric complex of gH/gL and three small glycoproteins, UL128, UL130 and UL131 (UL128-131), and appears to involve fusion at endosomal membranes. Strains of HCMV, such as AD169 and Towne, which have undergone extensive serial passage in cultured fibroblasts, fail to express the pentameric gH/gL/UL128-131 complex on virions and hence, are unable to infect epithelial and endothelial cells. Repair of a frameshift mutation in the UL131 gene of strain AD169, however, restores expression of gH/gL/UL128-131 and expands its cell tropism.

### SUMMARY

Wherefore, it is an object of the present invention to overcome the above mentioned shortcomings and drawbacks associated with the prior art The inventors observed that less extensively passaged HCMV strains that retain expression of gH/gL/UL128-131 can efficiently infect epithelial and endothelial cells. Nonetheless, several such strains replicate to ˜1000-fold lower titers on epithelial cells than does strain AD169 repaired for UL131. AD169 lacks a ˜15 kb region at the end of the unique long genome region, termed the ULb′. The inventors were intrigued by the rather striking differences in cell tropism between a laboratory strain AD169 repaired for expression of the pentameric gH/gL/UL128-UL131 complex, and strains, such as TB40/E, which have largely intact ULb′ regions and maintain expression of gH/gL/UL128-131. The inventors therefore hypothesized that the ULb′ region encoded an additional factor involved in HCMV cell tropism. The inventors' studies addressing this hypothesis led the inventors to identify a new function for UL148, a gene within the ULb′. The inventors discovered UL148 encodes an endoplasmic reticulum (ER) resident glycoprotein that influences virion cell tropism by regulating the composition of alternative gH/gL complexes.

Viral glycoproteins mediate entry of enveloped viruses into cells and hence play crucial roles in infection. In herpesviruses, a complex of two viral glycoproteins, gH and gL (gH/gL), regulates membrane fusion events and influences virion cell tropism. Human cytomegalovirus (HCMV) gH/gL can be incorporated into two different protein complexes: a glycoprotein O (gO)-containing complex, gH/gL/gO, and a complex containing UL128, UL130 and UL131, gH/gL/UL128-131. Variability in the relative abundance of the complexes in the virion envelope correlates with differences between HCMV strains in cell tropism. Nonetheless, the mechanisms underlying such variability have remained unclear. The inventors have identified a HCMV protein, UL148 encoded by the UL148 open reading frame of the HCMV genome (UL148) which influences the ratio of gH/gL/gO to gH/gL/UL128-131 and the cell tropism of HCMV virions.

A mutant disrupted for UL148 by the inventors showed defects in gH/gL/gO maturation and enhanced infectivity for epithelial cells. Accordingly, reintroduction of UL148 into an HCMV strain that lacked the gene resulted in decreased levels of gH/gL/UL128-131 on virions, and correspondingly, a decrease in infectivity for epithelial cells. UL148 localized to the endoplasmic reticulum, but not to the cytoplasmic sites of virion envelopment. Co-immunoprecipitation results indicated that gH, gL, UL130 and UL131, but not UL128 or gO, associate with UL148. Taken together, the findings suggest that UL148 modulates HCMV tropism by regulating the composition of alternative gH/gL complexes.

The entry of a virus into a cell is a fundamental step during infection. In certain herpesviruses, including Epstein-Barr virus, HHV-6, and human cytomegalovirus (HCMV), a viral glycoprotein complex called gH/gL plays key roles in entry and is found in two different forms on virions. The relative abundance of the two different types of gH/gL complexes is influenced by the type of cell from which the virus is produced, and influences the tropism of the virus for different cell types. The inventors have identified a viral glycoprotein, UL148, which influences the cell tropism of HCMV virions by regulating the relative amounts of these two gH/gL complexes. The findings have implications for understanding how herpesviruses navigate through host tissues. Additionally, based on these findings, the long sought goal of preparing effective vaccines against HCMV is arguably attainable through one of the vaccine preparation techniques known in the art, but with the added step of deleting, substituting, and/or modifying the UL148 gene and/or interfering with or modifying expression of the UL148 gene.

Recognizable homologs of UL148 appear to be conserved only among primate cytomegaloviruses. Nonetheless, the inventors consider it likely that other beta and gamma herpesviruses may express ER-resident glycoproteins that influence the composition of alternative gH/gL complexes on their virions in a manner analogous to that of UL148. Therefore, mutation or modification of genes from other herpesvirus genes that encode proteins functionally analogous to UL148 may be useful for obtaining effective vaccines against other beta and gamma herpesviruses.

In the present invention apparatus and methods are provided for changing a tropism of a herpes virus comprising the steps of one of deleting, substituting, or modifying a UL148 gene and interfering with or modifying an expression of the UL148 gene, optionally including where the herpes virus is human cytomegalovirus, and where the change in tropism enhances epithelial cell tropism, and where the epithelial cell line is ARPE-19.

In the present invention apparatus and methods are provided for increasing a ratio of gH/gL/UL128-131 to gH/gL/gO in a herpes virus comprising the steps of one of deleting, substituting, or modifying a UL148 gene and interfering with or modifying an expression of the UL148 gene, optionally including where the herpes virus is human cytomegalovirus.

In the present invention apparatus and methods are provided for preparing a vaccine for a herpes virus comprising the steps of one of deleting, substituting, or modifying a UL148 gene and interfering with or modifying an expression of the UL148 gene, optionally including where the herpes virus is human cytomegalovirus.

In the present invention apparatus and methods are provided for live virus vaccines to induce antibodies against the Pentamer.

In the present invention apparatus and methods are provided for protein based vaccines of recombinant Pentamer.

In the present invention apparatus and methods are provided for biologically produced (recombinant) monoclonal antibodies targeting the Pentamer, which can be used as infusions.

In the present invention apparatus and methods are provided for the man made organism TB_148^{HA}, the man made organism TB_Δ148, the man made organism ADr131_UL148^{HA}, the man made organism TB_148_{STOP}, and the man made organism TR_148_{STOP}.

In the present invention apparatus and methods are provided for preparing a vaccine for immunization against a herpes virus, comprising the steps of obtaining a solution containing either herpes viruses or an infectious herpesvirus genome cloned in Escherichia coli as a bacterial artificial chromosome (BAC), deleting, substituting, or modifying a UL148 gene of the human cytomegalovirus (human herpesvirus 5) or of a functionally analogous gene of any beta or gamma herpes virus in which alternative forms of gH/gL complexes are found on virions, and interfering with or modifying an expression of the UL148 gene of the herpes virus; using permissive cells to cultivate the herpes virus and/or to reconstitute an infectious virus from BAC DNA, micro-filtering the herpes virus solution to remove blood cells and other larger particles or impurities while letting the herpes virus pass through, and diluting the filtrate containing the herpes virus with a sterile saline solution, thereby forming vaccine, optionally including killing, attenuating, or otherwise inactivating the herpes virus.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIGS. 1A-1C are a pair of line graphs showing Titer, log₁₀ of for a range of days post infection for fibroblasts and epithelial cells each infected with TB_WT, TB_148^{HA}, or TB_Δ148 (FIG. 1A), a pair of bar graphs showing relative infectivity for TB_WT, TB_148^{HA}, and TB_Δ148 (FIG. 1B), and electrophoreses of lysates of TB_WT, TB_148^{HA}, and TB_Δ148 under non-reducing conditions and reducing conditions (FIG. 1C);

FIGS. 2A-2C are electrophoreses of lysates of TB_WT, TB_148^{HA}, and TB_Δ148 under non-reducing conditions and reducing conditions (FIG. 2A), Western blot of TB_148^{HA} and TB_Δ148 incubated in the presence of Endo H, PNGase F or buffer lacking an enzyme (FIG. 2B), and nine confocal microscopy images (FIG. 2C);

FIGS. 3A and 3B are a Western blot of ADr131_luc and ADr131_148^{HA} (FIG. 3A), and two bar graphs of relative infectivity of ADr131_luc and ADr131_148^{HA} (FIG. 3B);

FIGS. 4A and 4B are Western blots of Anti-HA IPs from lysates of TB_Δ148 and TB_UL148^{HA} infected fibroblasts (FIG. 4A), and of lysates of TB_UL148^{HA} infected fibroblasts prepared using anti-gH monoclonal antibody or a control IgG;

FIG. 5 is a model for the regulation of alternative gH/gL complexes by UL148;

FIGS. 6A-6E are a schematic representation of the construction of TB_Δ148 and TB_UL148^{HA} (FIG. 6A), electrophoreses of TB_WT, TB_Δ148, and TB_UL148^{HA} BACs digested endonucleases (FIG. 6B), a schematic representation of the predicted topology of UL148 (FIG. 6C), a Western blot of fibroblasts infected with TB_Δ148, and TB_UL148^{HA} (FIG. 6D), and a bar graph showing relative mRNA levels of various genes for fibroblasts infected with TB_WT, TB_Δ148, and TB_UL148^{HA} (FIG. 6E);

FIG. 7 is a set of six differential interference contrast microscopy images for epithelial cells infected with TB_WT, TB_Δ148, and TB_UL148^{HA}, three and thirteen days post infection;

FIGS. 8A-8D are a schematic representation of the procedure to determine differences in epithelial tropism (FIG. 8A), two bar graphs of validation of DNase treatment of virons (FIG. 8B), a schematic representation of a procedure for preparation of Western blot of pellet virons (FIG. 8C), and a Western blot comparison of viral glycoprotein expression in cell lysates and virons (FIG. 8D);

FIGS. 9A and 9B are three bar graphs showing relative infectivity of TB_WT, TB_Δ148, and TB_UL148^{HA} (FIG. 9A), and three bar graphs showing relative infectivity of ADr131_luc and ADr131_148^{HA} (FIG. 9B);

FIGS. 10A and 10B are a bar graph of relative infectivity of TB_WT, TB_Δ148, and TB_UL148^{HA} (FIG. 10A), and a blot of cell lysates from cells in FIG. 10A (FIG. 10B);

FIGS. 11A and 11B are a bar graph showing relative intensity for gH/gL/gO for TB_WT, TB_Δ148, and TB_UL148^{HA} (FIG. 11A), and two bar graphs showing relative intensity for gH and for gO for TB_Δ148, and TB_UL148^{HA} (FIG. 11B);

FIG. 12 is a Western blot of lysates of fibroblasts infected with either TB_UL148^{HA} or TB_Δ148 and incubated in the presence of Endo H, PNGase F, or in buffer lacking an enzyme;

FIG. 13A-13D is a schematic representation of ADr131_Luc and ADr131_148^{HA} BACs (FIG. 13A), electrophoreses of restriction digests of ADr131_Luc and ADr131_148^{HA} BACs (FIG. 13B), a Western blot of cell lysates of fibroblasts infected with ADr131_Luc or ADr131_148^{HA} (FIG. 13C), and a line graph showing multi-cycle replication kinetics of ADr131_Luc and ADr131_148^{HA} on fibroblasts (FIG. 13D);

FIG. 14 is a schematic illustration that demonstrates the effect of deleting, removing, or otherwise interfering with expression of UL148 from the HCMV virus;

FIG. 15 is a top portion of a schematic illustration of a strategy used in UL148 “STOP” viruses;

FIG. 16 is a is a bottom portion of a schematic illustration of a strategy used in UL148 “STOP” viruses that prevented expression of the UL148 protein by replacing all in-frame ATG “start” codons (methionine codons) with DNA encoding stop codons (e.g. TAG, TGA, TAA; which in RNA form are UAG, UGA, UAA);

FIG. 17 are a pair of electrophoreses showing “stop” viruses in HCMV strains TR and TB40/E, such as in FIG. 16, perform the same as the published deletion virus, that is, reducing levels of gH/gL/gO; and

FIG. 18 is a table of various primers used to detect various mRNAs, including for gH, gO, gL, UL128, UL130, and UL131, for example.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention.

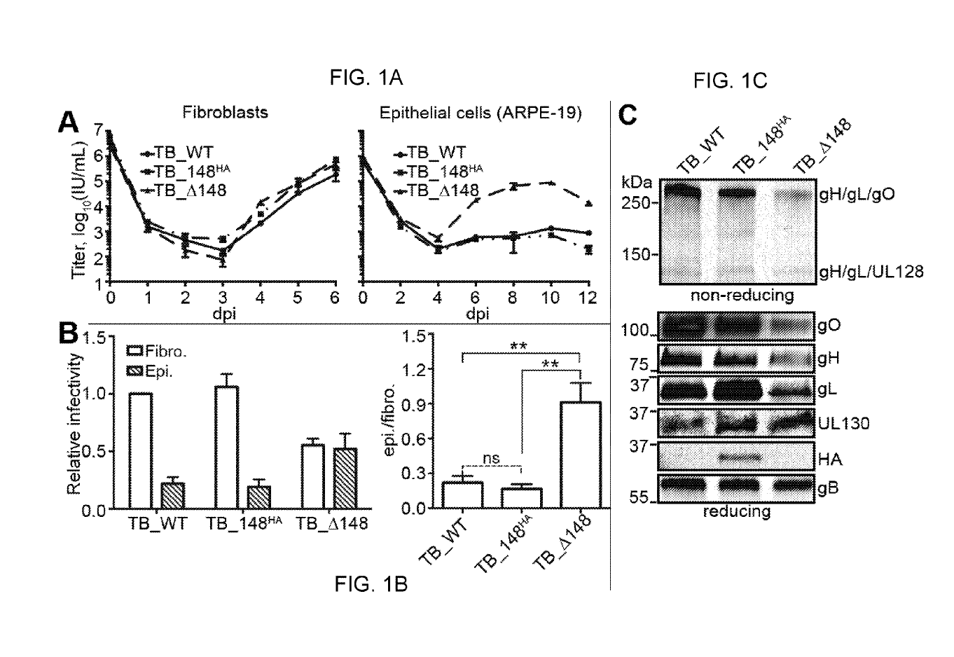

Turning now to FIGS. 1A-1C, a brief description concerning the various components of the present invention will now be discussed. As shown in FIG. 1, disruption of UL148 enhances infection of epithelial cells and alters the ratio of gH/gL complexes in strain TB40/E. FIG. 1A shows supernatants of cells infected at multiplicity of infection (MOI) of 1 were collected at indicated time points days post infection (dpi) and infectivity was determined; IU: infectious units. FIG. 1B shows virus preparations were measured by gPCR for genome copies/mL and tissue culture infectious dose 50 (TCID₅₀) was determined in parallel on human fibroblasts and ARPE-19 epithelial cells. TCID₅₀ results were normalized to viral genome copies and are compared relative to infectivity of WT virus on fibroblasts, which was set to 1.0. Results from three independent experiments are depicted. For both FIGS. 1A and 1B, error bars represent standard error of the mean (SEM). FIG. 1C shows lysates of glycerol-tartrate gradient purified virions that were electrophoresed under non-reducing conditions, and gH/gL/gO and gH/gL/UL128 complexes that were detected using a gH mAb (top panel). Duplicate aliquots of sample were treated with reducing agent and monitored for expression of the indicated proteins (bottom panel).

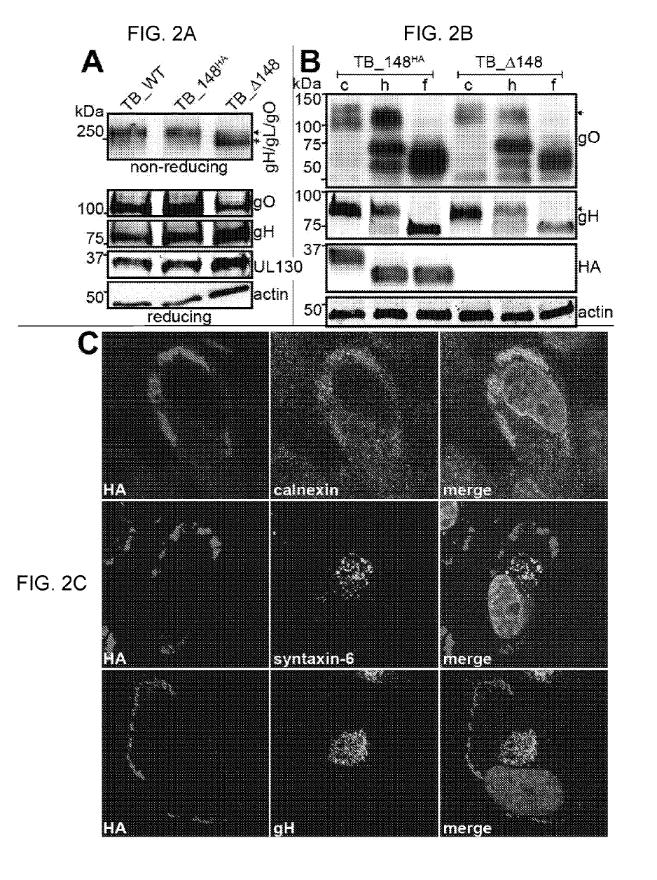

As shown in FIGS. 2A-2C, UL148 is an ER-resident glycoprotein that influences the maturation of gH and gO. FIG. 2A shows infected fibroblasts that were lysed at 72 hours post infection (hpi) and expression of the gH/gL/gO complex was determined under non-reducing conditions (upper panel). Duplicate aliquots were also assayed under reducing conditions for expression of gO, gH, UL130 and beta-actin (lower panel). FIG. 2B shows 72 hpi lysates of fibroblasts infected with TB_148^{HA} or its UL148-null derivative, TB_Δ148, were incubated in the presence of Endo H (h), PNGase F (f), or in buffer lacking enzyme (c), and subsequently assayed by Western blot. FIG. 2C shows fibroblasts infected with TB_148^{HA} were fixed at 72 hpi and imaged by confocal microscopy after staining with antibodies specific for: HA (red) to detect UL148; calnexin (green), an ER marker; syntaxin 6 (green), a TGN marker; and gH, as indicated. DAPI counterstaining of nuclei (blue) is shown in merged images (all colors refer to original colored images).

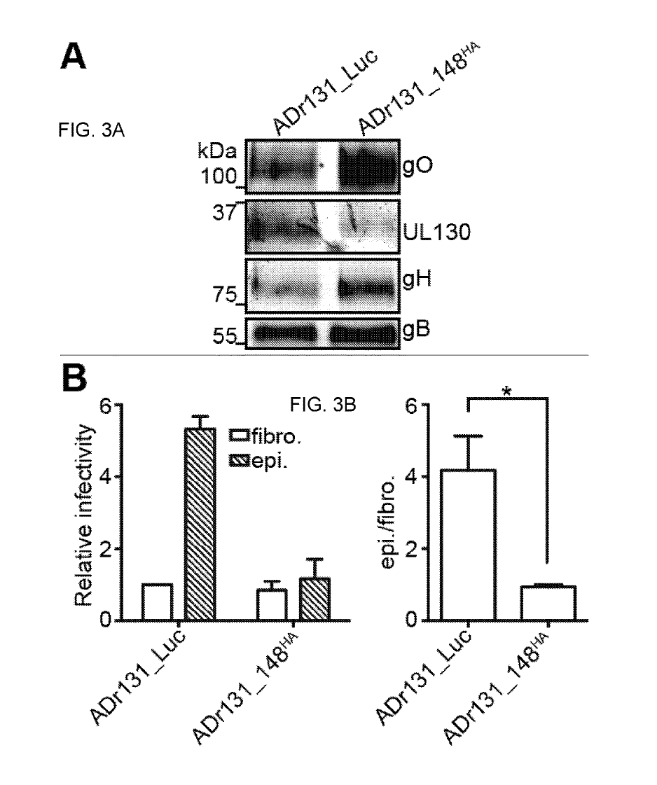

As shown in FIGS. 3A and 3B, introduction of UL148 to a laboratory-adapted HCMV strain is sufficient to influence the ratio of gH/gL complexes and cell tropism. Shown in FIG. 3A, purified virions were compared for the expression of gO, UL130, gH and gB, Shown in FIG. 3B, TCID₅₀ measurements were normalized to viral genomes per mL, and results for each condition are shown relative to those of ADr131_Luc on fibroblasts, which were set to 1.0. Error bars represent standard error of the mean (SEM). ns: not significant; *, P<0.05 by Student's t-test.

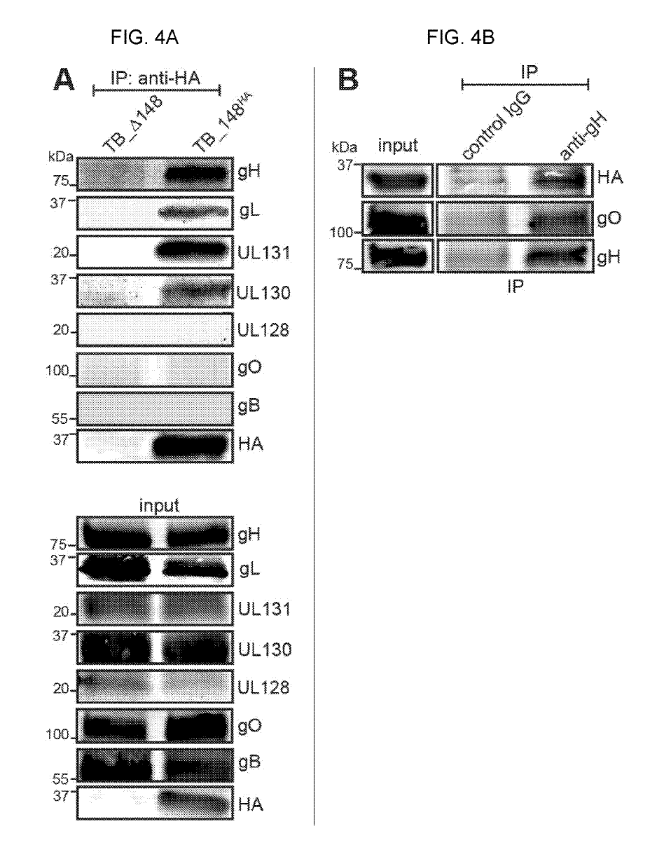

Turning now to FIGS. 4A and 4B, co-immunoprecipitation studies from infected cells are shown. FIG. 4A shows anti-HA IPs from lysates of TB_Δ148- and TB_UL148^{HA}-infected fibroblasts were assayed alongside input lysates for detection of gH, gL, UL128, UL130, UL131, gO and HA epitope. FIG. 4B shows lysates of TB_UL148^{HA}-infected fibroblasts were prepared using anti-gH monoclonal antibody or a nonspecific mouse IgG, eluted, and monitored alongside input lysates for detection of HA epitope, gO and gH.

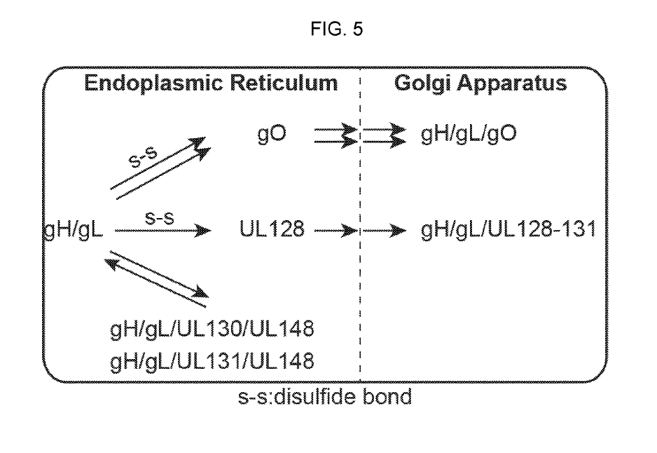

Turning now to FIG. 5, a model for the regulation of alternative gH/gL complexes by UL148 is shown. During the assembly of gH/gL complexes in the endoplasmic reticulum (ER), UL148 competes with UL128 for loading onto immature gH/gL complexes that contain either UL130 or UL131. The resulting gH/gL/UL130/UL148 and gH/gL/UL131/UL148 complexes are retained in the ER due to the RXR signal on the cytoplasmic tail of UL148. Because gO and UL128 form covalent, disulfide linkages (s-s) with gH/gL, while UL131 and UL130 do not, the ER-retained gH/gL/UL130 and gH/gL/UL131 complexes can dissociate to allow loading of gO. By competing for loading of UL128 onto gH/gL, UL148 selectively promotes the formation of gH/gL/gO complexes, which mature to the Golgi apparatus, and ultimately become available for incorporation onto the virion envelope.

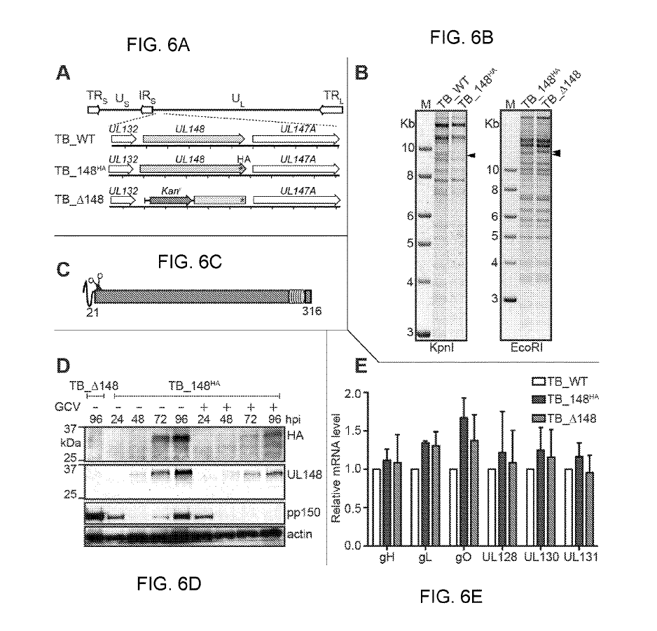

Turning now to FIGS. 6A-6E, construction and characterization of TB_Δ148 and TB_148^{HA} is shown. FIG. 6A shows a schematic representation of the construction of TB_148^{HA} and TB_Δ148 viruses. TB_UL148^{HA} and TB_Δ148 were constructed in the context of an infectious bacterial artificial chromosome (BAC) of strain TB40/E (TB40-BAC4). In TB_148^{HA} sequences encoding a C-terminal hemagglutinin tag (YPYDVPDYA) were incorporated at the 3′ end of the open reading frame encoding UL148. In TB_Δ148, a portion of UL148 coding region in TB_UL148^{HA} was replaced with a kanamycin resistance allele (Kan^{r}). The deleted residues correspond to nucleotide positions 211973 through 212439 of the TB40/E BAC GenBank accession number EF999921.1. FIG. 6B shows analysis of TB_148^{HA} and TB_Δ148 BACs by endonuclease digestion. TB_148^{HA} BAC and parental TB_WT BAC were digested with KpnI, and TB_Δ148 and TB_148^{HA} BACs were digested by EcoRI. Digested samples were analyzed by agarose gel electrophoresis. Lane M, molecular size marker. Arrowheads indicate predicted differences. FIG. 6C shows the predicted topology of UL148. A signal peptide (comprising aa 1-20) is represented by a black serpentine line, the predicted signal peptidase cleavage site between as 20-21 is represented by a pair of scissors, a 265 as ectodomain, spanning from as 21-285, is represented as a green rectangle, a 23 as transmembrane helix is shown as yellow rings, and an 8 aa cytoplasmic tail is indicated by a blue rectangle. FIG. 6D shows UL148 is expressed with leaky-late kinetics. Fibroblasts were infected for 2 h with TB_148^{HA} or TB_Δ148. Upon removal of inocula, infected cells were the incubated in medium containing 15 μM ganciclovir (GCV) to inhibit viral DNA synthesis. Cell lysates were collected at indicated time points, and subjected to Western blotting. The following antibodies were used to detect proteins: rabbit anti-HA, rabbit anti-UL148, mouse anti-pp150, and mouse anti-β-actin. FIG. 6E shows analysis of mRNA levels for gH, gL, gO, UL128, UL130 and UL131. Fibroblasts were infected with TB_WT, TB_148^{HA} or TB_Δ148, and harvested for total RNA at 72 hpi. RNA was reverse-transcribed with oligo (dT) to generate cDNA, which was used as template to quantify the cDNA level of indicated viral genes by quantitative PCR. The mRNA level from WT infection was set to 1. The average results from two independent biological replicates are depicted. Error bars indicate standard error of the mean (SEM).

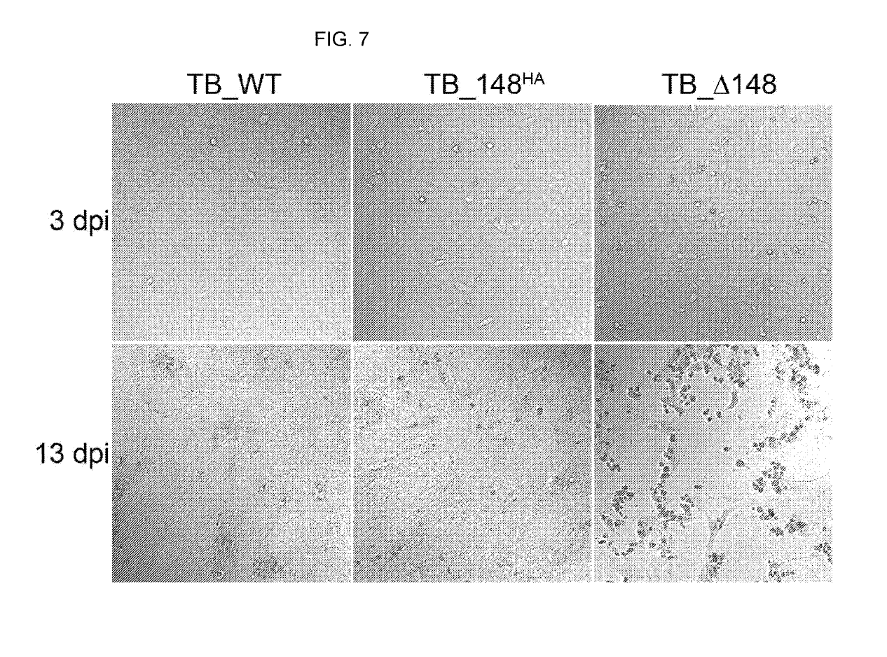

As shown in FIG. 7, a virus disrupted for UL148 shows enhanced cytopathic effect during infection of epithelial cells. ARPE-19 epithelial cells were infected with the indicated viruses at MOI 1 (as per titer on fibroblasts) while still subconfluent. Differential interference contrast microscopy was used to capture images of infected monolayers at 3 and 13 days post infection (dpi).

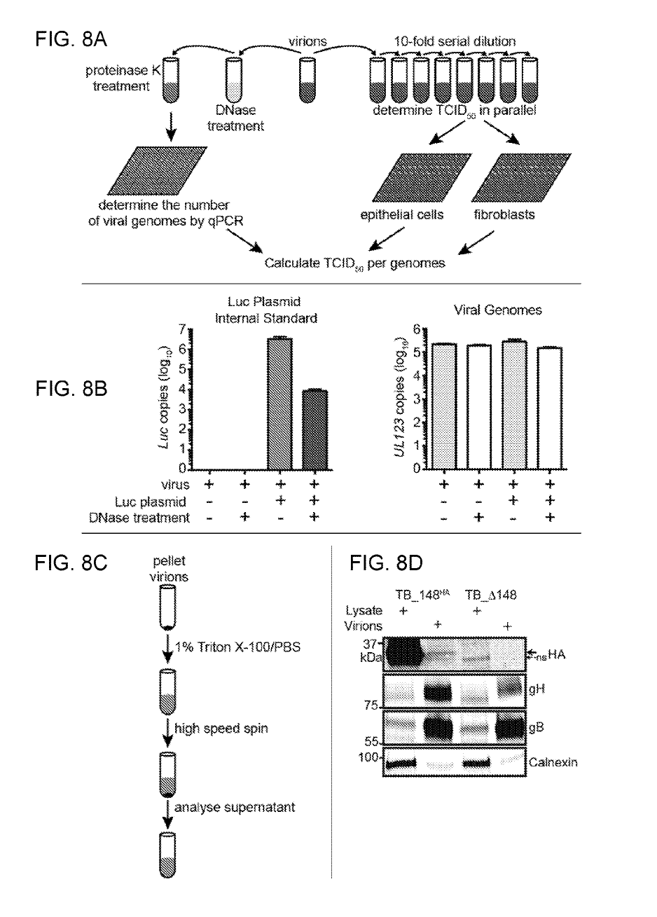

Turning now to FIGS. 8A-8D, analysis of virions is shown. FIG. 8A shows a schematic representation of the procedure to determine differences in epithelial tropism. Serial dilutions of the viral stocks to be compared are applied in parallel in 96 well cluster plates containing either ARPE-19 or human foreskin fibroblast cells (HFF). Infected wells are identified by staining at 24 hpi using a monoclonal antibody specific for the viral nuclear antigen IE1, and a TCID₅₀ is calculated. In parallel, the number of viral genomes in 10 μL of virion stock solution is determined by first treating with DNAse to degrade any non-enveloped viral DNA. Packaged viral DNA is then released by incubating in a detergent containing buffer containing proteinase K. Viral DNA is then precipitated and resuspended, and viral genome copies are determined using qPCR. Additional details are provided in below. FIG. 8B shows RNase treatment of virions was validated to decrease by ˜400-fold detection of a control plasmid (“Luc plasmid,” pGL3 basic, Promega, Inc.) that was spiked into a virion samples by DNAase treatment. FIG. 8C shows a schematic representation of the procedure for detergent extraction of viral glycoproteins from virions to prepare samples for Western blot analyses, as described below. FIG. 8D shows a comparison of viral glycoprotein expression in cell lysates and virions. HFF were infected with the indicated viruses at MOI=1 and cell lysates were harvested at 144 hpi. Separately, virions from infected cell supernatants were pelleted by ultracentrifugation through a 20% sorbitol cushion, resuspended in PBS containing 1% Triton X-100 detergent and spun down at 20,800×g for 30 min; the soluble (supernatant) fraction was then loaded such that gB signal would be consistent between samples. Antibodies against HA, gH, gB and calnexin were used for detection in Western blots. “ns”: non specific band, as indicated by dotted arrow. The solid arrow indicates the anti-HA immunoreactive band interpreted to represent UL148.

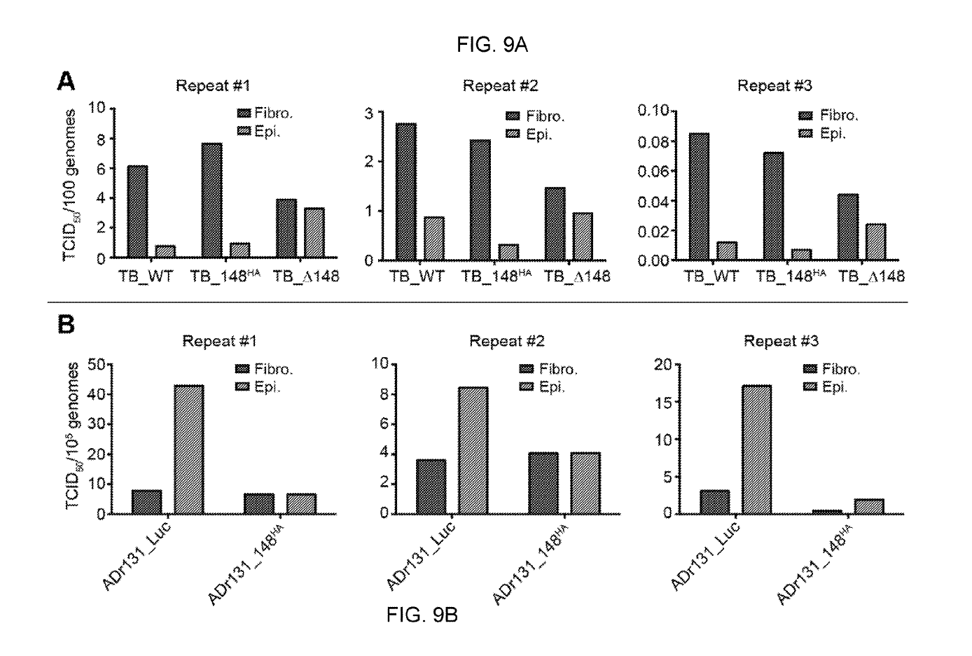

Turning now to FIGS. 9A and 98, raw data for FIGS. 1B and 3B are shown. Absolute infectivity results for each independent biological replicate of the indicated experiments are shown. FIG. 9A shows the results of the three independent biological replicates used to generate relative infectivity results shown in FIG. 1B. FIG. 9B shows the results of the three independent biological replicates used to generate relative infectivity results shown in FIG. 3B.

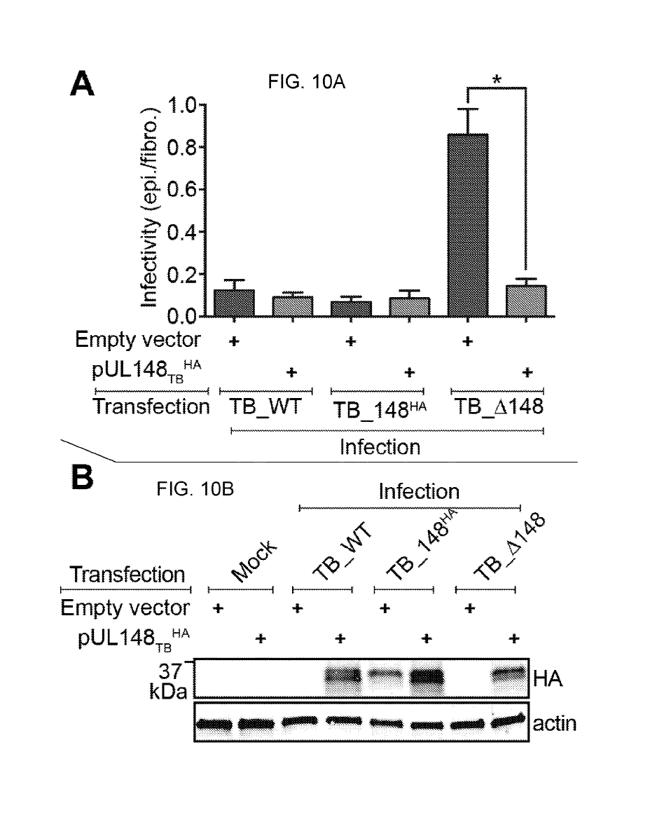

As shown in FIGS. 10A and 10B, UL148 expression in trans reverses the tropism phenotype of TB_Δ148. FIG. 10A shows HFF were nucleofected with pUL148_{TB}^{HA} or empty vector, as a control. At 6 hours post nucleofection, the HFF were either infected with TB_WT, TB_148^{HA}, TB_Δ148, or mock-infected, as indicated. Supernatants were collected at 144 hpi and infectivity in the TCID₅₀ normalized to viral genomes was determined, in parallel on HFF and ARPE-19. The data shown are from three independent biological replicates. Error bars represent standard error of the mean (SEM). *, P<0.05. FIG. 10B shows the 144 hpi cell lysates from 10A were harvested and blotted with rabbit anti-HA and mouse anti-β-actin antibodies.

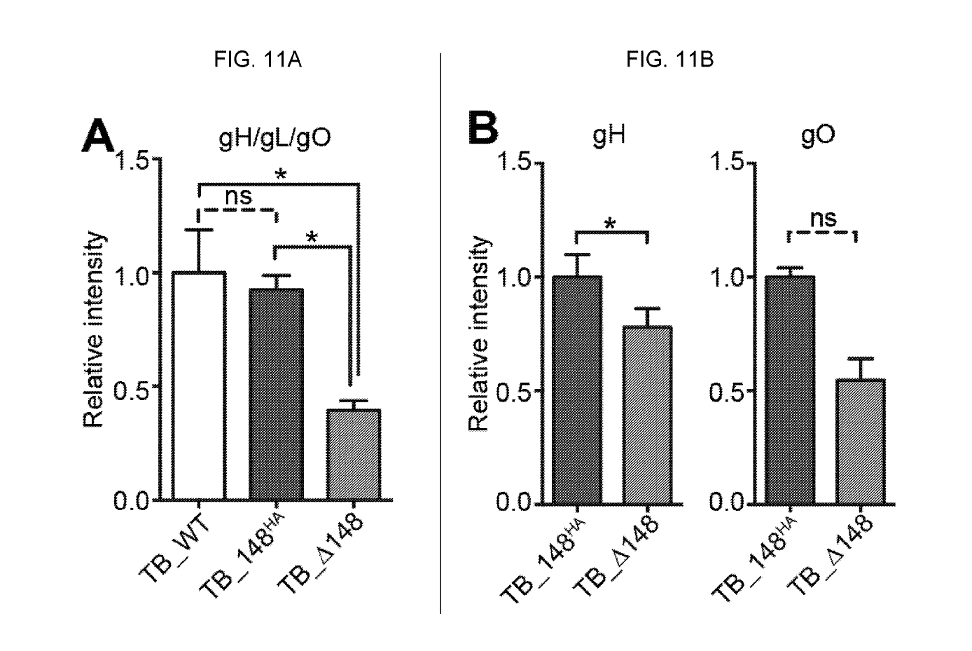

Turning now to FIGS. 11A and 11B, Quantification of Western blots in FIG. 1C and FIG. 2B are shown. A Li-Cor Odyssey system was used to quantify results of three independent biological replicates for each of the experiments shown in FIG. 1C (upper panel) and FIG. 2B. FIG. 11A shows signal intensity for gH/gL/gO was normalized to signal intensity from detection of gB. To facilitate comparisons, results were converted to “Relative Intensity” in that they are shown relative to those for TB_WT, which was set to 1; ns, not significant; Error bars represent standard error of the mean (SEM). *, P<0.05 by an ordinary one-way analysis of variance (ANOVA) with Tukey's post-test. FIG. 11B shows signal intensity for Endo H-resistant gO and gH species were measured relative to total signal intensity for the entire lane. To facilitate comparisons, results were converted to “Relative Intensity” such that they are shown relative to those for TB_148^{HA}, which were set to 1, ns, not significant; *, P<0.05 by the Student's t-test. Error bars represent SEM.

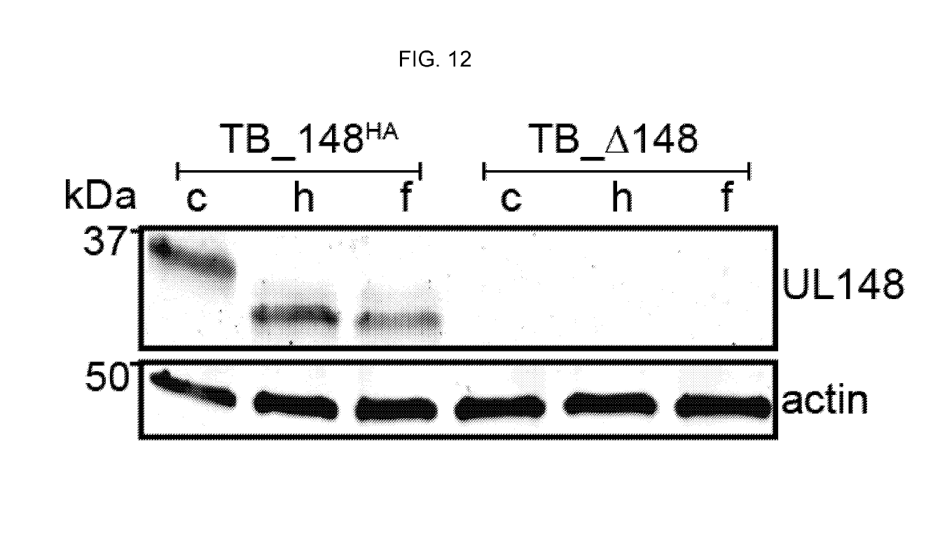

As shown in FIG. 12, UL148 is sensitive to Endo H treatment. 72 hpi lysates of HFF infected with either TB_148^{HA} or TB_Δ148 were incubated in the presence of Endo H (h), PNGase F (f), or in buffer lacking enzyme (c), and subsequently assayed by Western blot using anti-UL148 rabbit antisera.

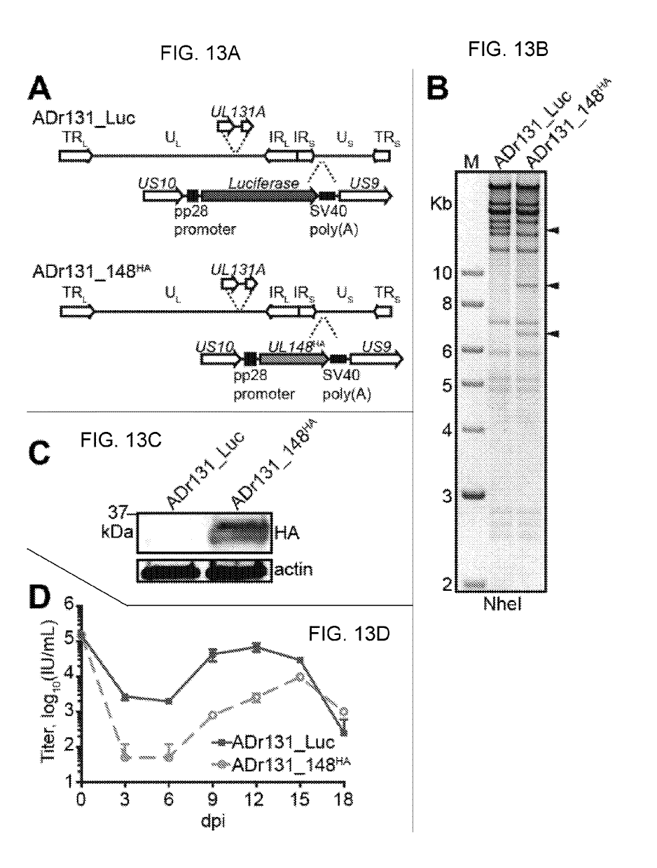

Turning now to FIGS. 13A-13D, construction of a laboratory strain AD169 restored for UL148 is shown. FIG. 13A shows a schematic representation of ADr131_Luc and ADr131_148^{HA} BACs. ADr131_148^{HA} encodes an HA-tagged UL148 in place of the firefly luciferase (Luc) open reading frame found in ADr131_Luc. In both viruses, an extra copy of the pp28 promoter drives expression of the introduced gene in the synthetic expression cassette found between Uₛ9 and Uₛ10. The repaired UL131 allele is indicated by a pair of rightward facing arrows connected by a short line to represent the two exons and intron of UL131. FIG. 13B shows restriction digest of ADr131_Luc and ADr131_148^{HA} BACs. BAC DNAs were digested using Nhel and resolved by agarose gel electrophoresis; M: DNA size ladder. Arrowheads indicate expected changes in the restriction fragmentation patterns. FIG. 13C shows detection of UL148 from ADr131_148^{HA} infected fibroblasts. HFF were infected with ADr131_Luc or ADr131_148^{HA}, respectively. Cell lysates were harvested at 120 hpi, and analyzed by Western blot using rabbit anti-HA and mouse anti-β-actin antibodies. FIG. 13D shows multi-cycle replication kinetics for ADr131_Luc and ADr131_148^{HA} on HFF. HFF were infected at MOI 0.05, and for each of the indicated time points, samples containing combined cell-free and cell-associated virus were measured for infectivity on HFF. IU/mL: infectious units per mL. Error bars indicate standard error of the mean (SEM).

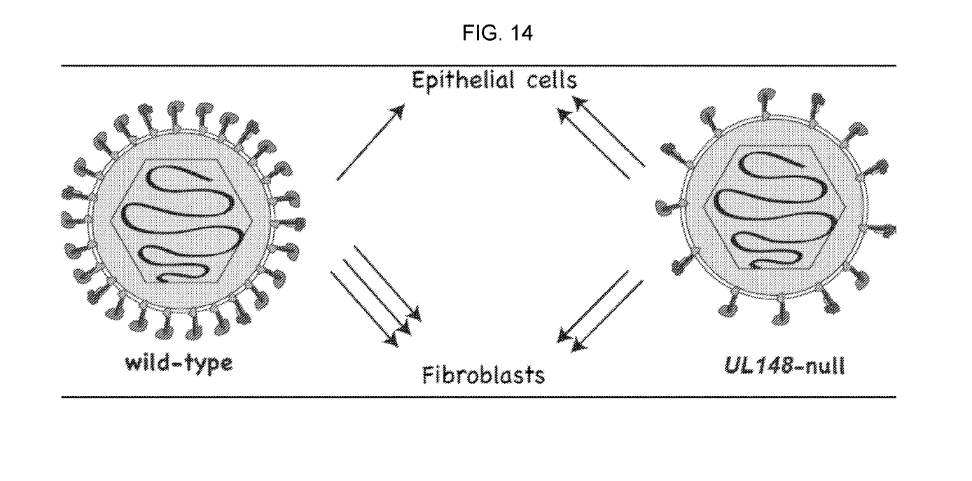

Turning now to FIG. 14, a schematic representation of the effect of deleting, removing, or otherwise interfering with expression of UL148 from the virus is shown. An increased infection of epithelial cell types, and somewhat decreased infection of fibroblasts for the virus without UL148 because that virus has less gH/gL/gO (Trimer) on its surface. The other version of gH/gL in HCMV is gH/gL/UL128-131 (Pentamer). Pentamer is necessary for the virus to enter epithelial cells, but not fibroblasts. Importantly, there is significant evidence that antibodies against Pentamer can protect women from transmitting the virus to the developing fetus, but antibodies against Trimer do not.

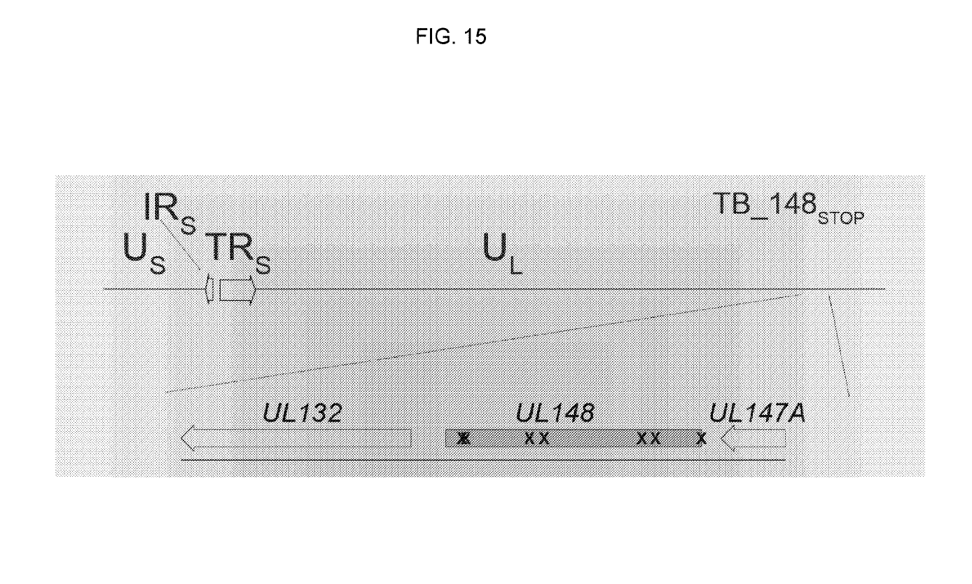

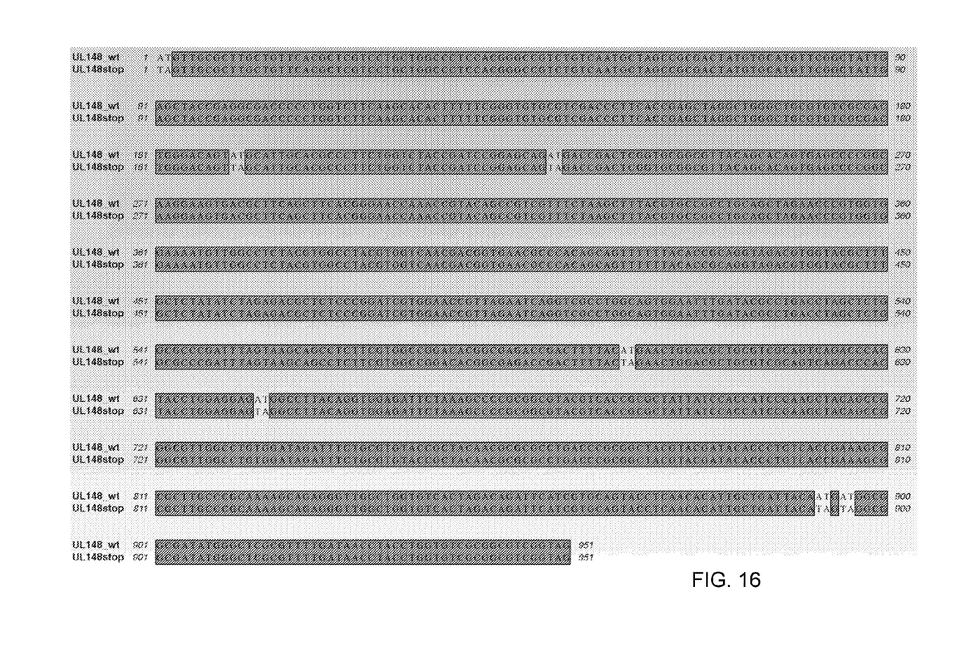

Turning now to FIGS. 15 and 16, these figures illustrates a further strategy used in UL148 “STOP” viruses. For these viruses, the inventors prevented expression of the UL148 protein by replacing all in-frame ATG “start” codons (methionine codons) with DNA encoding stop codons (e.g. TAG, TGA, TAA; which in RNA form are UAG, UGA, UAA),

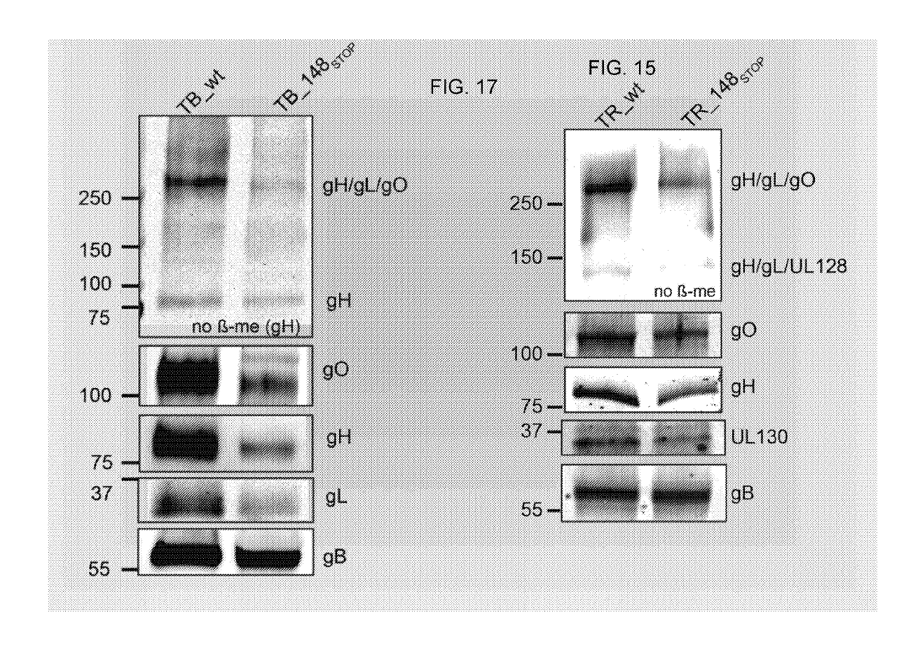

Turning now to FIG. 17, functionality of the “stop” viruses, described in FIGS. 15 and 16, in HCMV strains TR and TB40/E are shown to reduce levels of Trimer (gH/gL/gO) expression just as the deletion viruses described above.

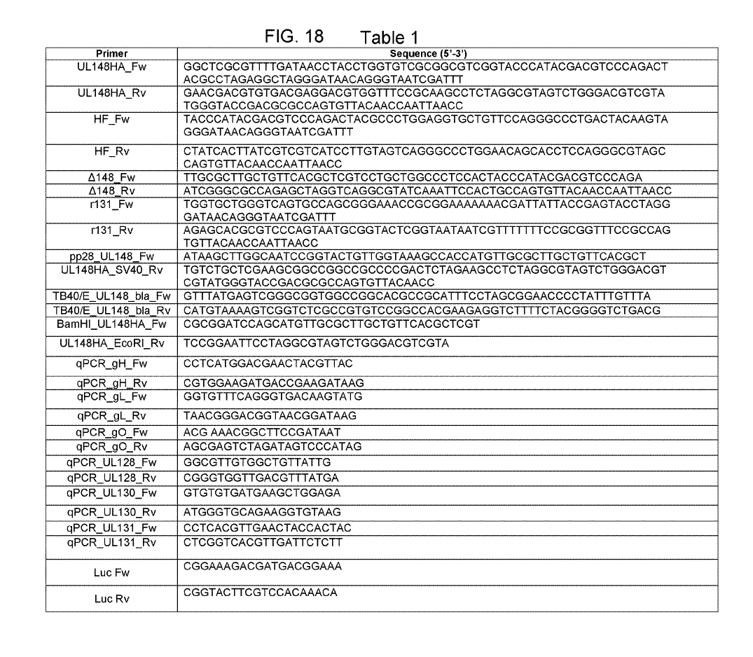

Turning to FIG. 18, Table 1 is shown. This table contains primers used in the disclosure herein.

To determine whether the HCMV UL148 gene encoded a protein that influenced virion cell tropism, the inventors constructed two recombinant viruses based on an infectious bacterial artificial chromosome (BAC) clone of HCMV strain TB40/E (Sinzger), and the two strains were dubbed TB_148^{HA} and TB_Δ148. TB_148^{HA} is a derivative of the wild-type TB40/E. (“TB_WT”) that expresses an influenza hemagluttinin epitope (HA) tag at the C-terminus of UL148. TB_Δ148 is a derivative of TB_148^{HA}, in which a large portion of UL148, comprising most of the 5′ half of the gene, was deleted. A ˜35 kD protein, which was immunoreactive to both anti-HA antibodies and to a polyclonal antisera raised against a synthetic peptide matching UL148 residues 263-285, was detected from cells infected with TB_148^{HA}, but not from cells infected with TB_Δ148. The protein was expressed with leaky late kinetics, and was interpreted to be the protein encoded by UL148.

The inventors interpreted this protein to be encoded by UL148. For simplicity, this protein may be referred to in this disclosure as simply “UL148.”

As shown in FIG. 1A, during MOI of 1 infection of human foreskin fibroblasts (HFF), the replication of TB_Δ148 was indistinguishable from TB_WT and TB_148^{HA}. However, in ARPE-19 human retinal pigment epithelial cells, TB_Δ148 replicated to 100-fold higher titers than TB_148^{HA} or TB_WT, and, as shown in FIG. 7, caused markedly enhanced cytopathic effects. As shown in FIGS. 1B, 8D, and 9A, when TB_Δ148, TB_WT and TB_148^{HA} were compared side-by-side in HFF and ARPE-19 cells for the ability to enter and initiate viral gene expression, TB_Δ148 virions showed approximately equivalent infectivity for HFF and ARPE-19 cells, while those of TB_WT and TB_148^{HA} showed 5-fold higher infectivity for HFF than for ARPE-19. As shown in FIGS. 10A and 10B, when UL148 was expressed in trans prior to infection with TB_Δ148, the tropism of progeny virions was restored to that seen for TB_WT, which suggests the tropism phenotype of TB_Δ148 was due to the absence of the protein encoded by UL148.

Returning to FIGS. 6A-6E, the protein encoded by UL148 was predicted to harbor a signal peptide at the N-terminus, which would be cleaved, leaving a 265 aa ectodomain anchored by a 23 aa transmembrane helix that terminates in a short, 8 aa cytoplasmic tail. Nonetheless, it was initially unclear how the protein might influence HCMV replication in epithelial cells. Because alternative gH/gL complexes play important roles in cell tropism of HCMV, the inventors were curious whether the tropism phenotype of the TB_Δ148 might involve differences in their expression.

As shown in FIGS. 1C and 11A and 11B, using a gH specific monoclonal antibody (mAb) to detect gH/gL complexes resolved under non-reducing conditions, the inventors noted that a ˜300 kD band, which represents the mature gH/gL/gO complex, was approximately 2.5-fold less abundant in TB_Δ148 virions than in virions of TB_WT or TB_148^{HA}. Continuing with FIG. 1C, although TB40/E virions contain relatively low levels of UL128, a ˜135 kD band, which represents a disulfide-linked complex of gH/gL with UL128, was likewise detectable, albeit faintly. While levels of gB were consistent across all lanes, TB_Δ148 virions showed decreased levels of gO, gH, and gL, compared to TB_WT or TB_148^{HA}. To become incorporated into virions, UL130 first must assemble within the ER as a subunit of the complete gH/gL/UL128-131 complex; otherwise protein constituents of the gH/gL complexes fail to transit to the Golgi. The observation of similar UL130 levels across all three lysates thus suggests that virion levels gH/gL/UL128-131 were not strongly affected by disruption of UL148. Even though the inventors did detect UL148 in TB_148^{HA} virions, as shown in FIG. 1C, the bulk of UL148 remained within infected cells. Whereas, as shown in FIG. 8D, the opposite was true for gH and gB. While the significance of the virion-associated UL148 was interpreted with caution, the inventors' results suggested that disruption of UL148 led to decreased levels of gH/gL/gO in virions, and to enhanced tropism for ARPE-19 epithelial cells.

As shown in FIG. 6E, the inventors did not detect differences in mRNA levels for gH, gL, gO, or UL128-131 that could explain the tropism differences and effects on gH/gL expression that the inventors observed for TB_Δ148. However, as shown in FIG. 2A, the inventors found that gH/gL/gO from TB_Δ148-infected cells migrated more rapidly than that from TB_WT or TB_148^{HA} infections. gH/gL/gO forms a 220-kD complex within the ER, but only matures to the ˜300 kD form found on virions once it has reached the Golgi. To address whether these mobility differences might indicate differences in glycosylation, cell lysates of TB_Δ148 and TB_148^{HA} infections were incubated with endoglycosidase H (endoH), PNGase F, or buffer alone, and assessed for effects on protein mobility.

Turning to FIGS. 2B, 11A, and 11B, endoglycosidase treatment appeared to enhance overall detection of gO, perhaps because the anti-gO polyclonal antibody the inventors used was raised against a synthetic peptide, and hence may more efficiently recognize target epitope(s) in the context of partially- or fully deglycosylated protein. Nonetheless, levels of the slowest migrating, EndoH-resistant form of gO were nearly two-fold higher in TB_148^{HA} infected HFF, on average, than in TB_Δ148-infected HFF. Levels of slowest migrating form of gH were 1.3-fold higher, on average, in TB_148^{HA}-infected HFF compared to TB_Δ148-infected cells, and the difference was significant. EndoH is unable to remove highly branched oligosacharrides found on glycoproteins that traffic to the Gogli, while PNGase F removes all N-linked oligosaccharides. Thus, as expected, gO and gH from both lysates were fully sensitive to PNGase. These results were consistent with the possibility that gH/gL/gO maturation is delayed in the absence of UL148. Moreover, the HA-immunoreactive band, which the inventors interpreted to represent UL148, was fully sensitive to EndoH, as was a band immunoreactive to anti-UL148 polyclonal serum (FIG. 12), suggesting that UL148 is for the most part retained within ER during infection, as would be consistent with the presence of a putative RXR ER retention motif, RRR, in its predicted cytoplasmic tail, shown in FIG. 6C.

Turning to FIG. 2C, confocal microscopy studies of cells infected with TB_148^{HA}, anti-HA antibodies stained a semi-continuous ring-like structure, which co-localized with the ER marker calnexin. The anti-HA staining pattern did not overlap with the compartments stained by antibodies specific for syntaxin-6, a trans-Golgi network (TGN) marker, or by antibodies specific for gH, which instead co-stained a juxtanuclear structure, previously determined to be the cytoplasmic virion assembly compartment (cVAC), which is the site where newly formed virions acquire an infectious envelope. The inventors' results are consistent with the notion that the cVAC displaces ER from the side of nucleus on which it forms, and further suggest that UL148 is an ERresident glycoprotein.

The inventors next examined how UL148 expression would affect a laboratory adapted strain that otherwise lacks UL148. The inventors therefore constructed a third new recombinant virus, ADr131_148^{HA} from ADr131_Luc. The new recombinant virus harbors an intragenic cassette driving expression of UL148, and was repaired for the frameshift in UL131 to restore expression of gH/gL/UL128-131, as shown in FIG. 13A. Having confirmed that ADr131_148^{HA}-infected cells expressed UL148, the inventors compared gH/gL expression and tropism of ADr131_UL148^{HA} to its parental virus, ADr131_Luc, which harbors a luciferase gene instead of UL148. As shown in FIG. 3A, ADr131_148^{HA} virions showed increased levels of gO and gH, and decreased levels of UL130. Accordingly, as shown in FIG. 3B, ADr131_148^{HA} showed a ˜4-fold decrease in tropism for epithelial cells when compared to ADr131_Luc. However, despite the increased levels of gO, ADr131_148^{HA} virions did not exhibit increased infectivity for fibroblasts.

Turning to FIG. 3B, the differences in gH/gL expression and cell tropism between virions of ADr131_148^{HA} and and ADr131_Luc mirrored the differences the inventors observed between virions of a previously characterized AD169 repaired for UL131, BADrUL131, and those of strain TB40/E. The presence of UL148—whether in strain TB40/E or in ADr131_148^{HA} was associated with increased levels of gO and decreased levels of UL130, suggesting that UL148 can increase the abundance of the gH/gL/gO complex at the expense of the gH/gL/UL128-131 complex. As shown in FIG. 13D, ADr131_148^{HA} exhibited a 20-fold replication defect compared to parental ADr131_Luc. Shown in FIGS. 1A-1C, 3A, and 3B, these results suggested that roles for UL148 could in large part explain the differences in expression of gH/gL complexes and cell tropism between laboratory strain AD169 repaired for UL131 (e.g., BADrUL131) and strains with largely intact ULb′ regions, such as TB40/E, FIX, and TR (FIGS. 1A-1C, 3A, and 3B). Turning to FIGS. 4A and 4B, UL148 immunoprecipitates (IPs) were found to contain gH, gL, UL130, and UL131, but not gO, UL128, or gB. No proteins were detected from anti-HA IPs from TB_Δ148 infected cell lysates, which suggests the anti-HA IPs were specific for UL148 interacting proteins. Reciprocally, an anti-HA immunoreactive band matching the expected size of UL148 was detected in IPs for gH (FIG. 4B). The simplest interpretation of the co-IP results is that UL148 interacts with immature gH/gL complexes that contain either UL130 and/or UL131, but not gO or UL128.

Taken together, the inventors' findings identify UL148 as a virally-encoded factor that influences the cell tropism of at least a herpesvirus by regulating the composition of alternative gH/gL complexes on virions, likely through effects on maturation of the complexes as they assemble within or transit through the ER.

Despite the clear importance the inventors observed of alternative gH/gL complexes in virion cell tropism of several beta and gamma herpesviruses, the mechanisms that regulate their relative abundance during infection have for the most part remained elusive to prior researchers. The inventors' results evidence that HCMV makes use of virally encoded protein, UL148, to modulate the relative abundance on virions of two alternative gH/gL complexes by influencing their assembly and/or maturation, a finding that suggests a novel mechanism for regulation of virion tropism in a herpesvirus. Although, as shown in FIG. 1C, the inventors did detect UL148 in purified preparations of HCMV virions, as shown in FIG. 8D, the levels of expression in virions were extremely low compared to those found in cells. In contrast, as shown in FIGS. 2A-2C and 13C, UL148 was readily detected in the ER of infected cells, and displayed an endoglycosidase sensitivity profile consistent with ER-localizaton. Moreover, the presence of an RXR motif in the predicted cytoplasmic tail of UL148 provides a putative signal for ER-retention. Together, these observations suggest that UL148 exerts its influence on virion cell tropism via its effects on the maturation of gH/gL complexes.

EBV arguably provides the most well understood example for how gH/gL complexes, and hence virion cell tropism, are regulated in a herpesvirus. Class II HLA acts as a ligand for the EBV gH/gL accessory protein gp42, and because epithelial cells do not express HLA II, EBV virions produced from epithelial cells contain more gp42 and thus, more efficiently infect B-cells. B-cells, on the other hand, produce EBV virions with lower levels of gp42, apparently because gp42 interacts with HLA II molecules within the ER, reducing its expression on virions. Therefore, based on the inventors' findings, one might propose that EBV makes use of a cellular protein, HLA II, to regulate its expression of its alternative gH/gL complexes, while HCMV makes use of a viral protein, UL148, to do so. That both viruses utilize factors within the ER to regulate gH/gL complexes illustrates the relevance of the organelle as a foundry for determining the tropism of herpesvirus virions. Because the ER is where newly translated proteins begin their journey through the secretory pathway, factors within this organelle are well positioned to influence the repertoire of glycoproteins available for incorporation into the virion envelope.

Based on their findings shown in FIGS. 4A and 4B that UL148 interacts with gH/gL complexes containing UL130 and UL131, but not UL128 or gO, the inventors have proposed a mechanism to explain how UL148 influences the assembly of gH/gL complexes. As shown in FIG. 5, the inventors propose a mechanistic model in which that UL148 promotes the maturation of gH/gL/gO by occluding the formation of a disulfide bond between gH/gL and UL128. Because gO and UL128 have each been reported to form disulfide bonds to gH/gL, while UL130 and UL131 are not found to form disulfide bonds to gH/gL, the inventors reason that formation of a disulfide bond between UL128 and gH/gL would irreversibly commit a gH/gL dimer towards assembly into the pentanneric gH/gL/UL128-131 complex, while binding of UL130 and UL131 to gH/gL might interfere with loading of gO in a reversible manner. The inventors further reason that once bound to UL148, complexes of gH/gL with UL130 and/or UL131 could eventually dissociate, providing an opportunity for gO to load. Additional information supporting the model is that: (i) that UL128 and gO form disulfide bonds to the same cysteine residue on gL, (ii) that UL128, UL130 and UL131 can each bind independently to gH/gL, (iii) the presence of UL128 greatly increases the loading of UL130 and UL131, and (iv) gO and the UL128-131 proteins compete for binding of gH/gL.

Although the inventors found that HCMV deploys UL148 to regulate its virion gH/gL complexes, it would be premature to exclude the possibility that the regulation of cell tropism by HCMV might also involve cellular proteins,

With the inventors' results concerning UL148, described above, and the inventors' observation that UL130, UL131, gH, and gL co-immunoprecipitates with the protein, described above, the inventors conclude that an interplay exists between the UL128-UL131 proteins and UL148,

Methods And Materials

Cells and viruses. Primary human foreskin fibroblasts (HFF) were a gift of Jennifer Spangle and Karl Munger (Harvard Medical School, Boston, Mass.). Human retinal pigment epithelial cell line (ARPE-19, CRL-2302) was purchased from ATCC (Manassas, Va.). HFF and ARPE-19 cells were cultured in DMEM containing 10% fetal bovine serum, supplemented with gentamicin and ciprofloxacin. A BAC clone of HCMV strain TB40/E, TB40-BAC4 (TB40/E), was a gift of Christian Sinzger (University of Ulm, Ulm, Germany), the pp28_Luc BAC was provided by Donald Coen (Harvard Medical School, Boston, Mass.). BADrUL131-Y4 (BADrUL131) (Wang II) was a gift of Thomas Shenk (Princeton University, Princeton, N.J.).

HFF were cultured as described in Wang III. ARPE-19 retinal pigment epithelial cells were cultivated in the same media conditions used for HFF. TB40-BAC4, a BAC clone of HCMV strain TB40/E, was a gift of Christian Sinzger (Universitatsklinikum, Ulm, Germany). BADrUL131 was a generous gift of Tom Shenk (Princeton University, Princeton, N.J.). All other viruses were derived from TB40-BAC4 or pp28_Luc, Infectious virus was reconstituted from BAC DNA, propagated on HFF, concentrated by ultracentrifugation through 20% a sorbitol cushion, and measured for infectious units (IU) per mL, all as described in Wang III and Li II. Replication kinetics studies were conducted using infected cell supernatants, as described in Wang III and Li II. Glyceroltartrate gradient purification was performed as described in Chevillotte and Talbot.

Antibodies. The following primary antibodies were used in this study: mouse anti-HA antibody (#sc7392, Santa Cruz Biotech), rabbit anti-HA antibody (#A190-108A, Bethyl laboratories, Inc., Montgomery, Tex.), rabbit anti-calnexin (#2679, Cell Signaling Technologies), rabbit anti-syntaxin 6 (#1869, Cell Signaling Technologies, Danvers, Mass.), mouse anti-β-actin (926-42212, Li II-Cor, Inc., Lincoln, Nebr.). Secondary antibodies Alexa Fluor 488 goat anti-mouse IgG (#A11001), Alexa Fluor 488 goat anti-rabbit IgG (#A11008), Alexa Fluor 594 goat anti-mouse IgG (#A11012), Alexa Fluor 594 goat anti-rabbit IgG (#A11005), were purchased from Life Technologies, Inc. (Grand Island, N.Y.). Mouse anti-gB clone #27-156 and mouse anti-gH clone AP86 have been described in Wang I and Britt I. Rabbit anti-UL130 polyclonal serum (Britt II) was kindly provided by David C. Johnson (Oregon Health Sciences University, Portland, Oreg.). Rabbit antibodies to detect gO and gL have been described in Zhou.

BAC mutagenesis. TB_148^{HA} was generated as follows: Primers UL148^{HA}_Fw and UL148^{HA}_Rv, shown in Table 1 in FIG. 18, were used to amplify an excisable kanamycin resistance marker (Kan^{r}, otherwise known as “IScal-AphAI”. The PCR product was electroporated into E. coli strain GS1783 harboring the wild-type TB40/E BAC [TB40-BAC4]. Kan^{r} integrates were then resolved to yield the TB_148^{HA} BAC. TB_Δ148 was constructed as follows: HF_Fw and HF_Rv, shown in Table 1, were used to generate a PCR product, HF_Kanr, which was used as template in a second PCR reaction using primers Δ148_Fw and Δ148_Rv, shown in Table 1. The resulting product, which was designed to replace UL148 residues corresponding to nucleotide positions 211973 to 212439 of GenBank File Number EF999921.1, was electroporated into E. coli strain GS1783 harboring the TB_148^{HA} BAC, yielding TB_Δ148. ADr131_Luc was constructed as follows: an I-Scel-Kanr marker was amplified with primers r131_Fw and r131_Rv, shown in Table 1. The PCR product was electroporated into GS1783 harboring the pp28_Luc BAC. The Kan^{r} marker was removed “scarlessly,” by en passant mutagenesis, as described in Tischer 1 and Tischer 2, leaving behind a repaired UL131. The Kan^{r} intermediate BAC generated during construction of TB_148^{HA}, which contains an excisable I-Scel-Kan^{r} disrupting UL148, was used as a template in a PCR reaction using primers pp28_UL148_Fw and UL148^{HA}_SV40_Rv, as shown in Table 1. The resulting PCR product was electroporated into E. coli strain GS1783 harboring pp28_Luc BAC. Kanr integrates were resolved to yield the AD_148^{HA} BAC, which was then repaired for UL131 as described above. All newly constructed BACs were verified by DNA sequencing of the modified regions (Genewiz, Inc., South Plainfield, N.J.), and by restriction fragment pattern analysis.

Purification of virions. Fibroblasts were infected with the viruses at MOI of 1 and incubated for 6 days. Supernatants were collected, and virus was harvested and pelleted by ultracentrifugation through a 20% sorbitol cushion. Pelleted virions and associated particles were purified by glycerol tartrate gradient purification. Briefly, pelleted virions were resuspended by tituration in 40 mM sodium phosphate buffer (pH 7.4), layered onto a 35% to 15% glycerol-tartrate gradient in 14×89 mm Ultra-Clear™ tubes (Beckman Coulter, Inc., Brea, Calif.) and ultracentrifuged in a SW41 rotor at 23000 rpm for 45 min at 10° C., using slow acceleration and deceleration settings. For typical laboratory HCMV strains, such as AD169, the procedure produces three bands: an upper band consisting of non-infectious enveloped particles (NIEP), a middle band consisting of infectious virions, and a diffuse lower band representing dense bodies. In the inventors' experiments with strain AD169, which produces more abundant dense bodies, the lowest band was readily observed. In TB40/E infections, however, very few dense bodies are produced. Hence, during gradient purification of this strain, the lowest band was often difficult to observe. Virions (middle bands) were collected, pelleted at 22000 rpm at 5° C. for 1 h, resuspended in 40 mM sodium phosphate buffer, pH 7.4 and stored at −80° C. until use.

Quantification of viral genome copies. 10 μL aliquots of 144 hpi supernatant containing virus, or in the case of ADr131_148 vs ADr131_Luc, shown in FIG. 3B, purified virus preparations, were treated at 37° C. for 30 min using 1 μL RQ1 RNase-Free DNase I (Promega, Inc., Cat. #M6101), as per manufacturer's instruction, in a final reaction volume of 20 μL to degrade any viral DNA nonspecifically bound to the surface of virions. DNAse I digestion was stopped by adding 1 μL of RQ1 stop solution, and then incubating at 65° C. for 10 min. Virions were then lysed using 100 μL of lysis buffer (400 mM NaCl, 10 mM Tris, 10 mM EDTA, 0.2% SDS, 73 μg/mL proteinase K, pH 7.5) at 37° C. overnight to release virion genomic DNA. The reactions were then extracted twice using 200 μL of pH 8.0 buffered phenol chloroform solution (25 phenol:24 chloroform:1 isoamyl alcohol, EMD Millipore Cat #6805). 1 μL of 20 mg/mL glycogen, 10 μL of 3M sodium acetate pH 5.2, and 250 μL of 100% ethanol were then added to each reaction, and the tubes were incubated at −20° C. for 1 h to precipitate DNA. Reactions were then spun for 30 min at 12,000 RPM to pellet DNA. Pellets were washed with twice using 70% ethanol, dried, and resuspended in 50 μL of 10 mM Tris-HCl, 0.5 mM EDTA (TE). The number of viral genomes in 1 μL of the resuspended DNA material were quantified in duplicate real-time quantitative PCR reactions by detecting UL123 copies, including a standard curve for absolute quantification, as previously described in Wang III and Li II. The pGL3 Basic plasmid vector (Promega), which encodes Photinus pyralis luciferase, was added as an internal standard in the experiment shown in FIG. S3B to measure DNAase I degradation of non-specific DNA; qPCR primers to detect P. pyralis luciferase copies are shown in Table 1.

Plasmids. pUL148_{TB}^{HA}, a derivative of pEF1/V5-His C (Life Technologies, Inc.) that expresses UL148 under the Homo sapiens eukaryotic translation elongation factor promoter, was constructed as follows: BamHI_UL148^{HA}_Fw and UL148^{HA}_EcoRI_Rv were used in a PCR reaction with TB_148^{HA} BAC DNA as the template, shown in Table 1, the PCR product was treated with BamHI and EcoRI restriction enzymes, and then ligated to pEF1/V5-HisC plasmid (Life Technologies, Inc) that was linearized using the same enzymes. The plasmid was verified by DNA sequencing (Genewiz, Inc., South Plainfield, N.J.).

Transfection/Electroporation. HFF were transfected using an AmaxaR NHDF NucleofectorR Kit (Cat. VPD_1001, Lonza, Inc.) according to the manufacturer's protocol. Briefly, for each reaction, 5×105 freshly-trypsinized HFF were pelleted by centrifugation at 1000 rpm for 5 min, resuspended in a solution containing 3 μg plasmid premixed with 100 μL of Nucleofector™ Solution (82 μL of Nucleofector™ Solution and 18 μL of supplement). The cell suspension was then transfected using the U-023 program on a Nucleofector II (Lonza, Inc.), then plated and cultured by standard methods until infection.

Endo Hf and PNGase F treatment. Cell lysate was harvested at 72 hpi and treated with Endo Hf (Cat. #P0703S) or PNGase F (Cat. #P07045), each from New England Biolabs, Inc. (Ipswitch, Mass.), according to the manufacturer's instructions. Briefly, cell lysate was incubated with Glycogen Denaturing Buffer at 100° C. for 10 min, then incubated at 37° C. for 1 h in the presence of Endo Hf or PNGase F in the supplied buffer, or as a control, in G5 buffer lacking enzyme.

Immunofluorescence. HFF were seeded on Microscope Cover Glass (Cat. #12-545E, Fisher Scientific, Inc.), incubated until they reached ˜90% confluence, and then infected at an MOI 1. At 72 hpi, cells were washed with phosphate-buffered saline (PBS), consisting of 137 mM NaCl, 2.7 mM KCl, 10 mM Na₂HPO₄, 1.8 mM KH2PO4, pH 7.4, fixed for 15 min at room temperature in PBS containing 4% paraformaldehyde, washed in PBS, permeabilized for 4 min using 0.1% Triton X-100 (in PBS), washed in PBS, and blocked for 30 min at room temperature in PBS containing 5% goat normal serum (Cat ##23420, Rockland, Inc.). Cells were then incubated in the presence of primary antibodies for 1 h at 37° C., washed three times using PBS containing 0.1% Tween-20 (PBST) for 5 min per wash. The following Alexa Fluor (AF)-labeled goat polyclonal antibodies from Life Technologies were used for secondary detection: AF 488 anti-rabbit IgG (Cat #A11008), AF 594 anti-mouse IgG (A11012), AF 594 anti-rabbit IgG (Cat #A11005). Fluorescently labeled secondary antibodies were applied for 1 h at 37° C., after which cells were washed extensively in PBST. Cells were mounted using Prolong Gold antifade reagent containing 4′,6-diamidino-2-phenylindole (DAPI) (Cat ##P36931, Life Technologies, Inc.), which was used to counterstain nuclei. Images were captured using a Leica TCS SP5 Spectral Confocal Microscope running LAS AF software.

Western blotting, Western blotting, including quantification of signal from dye-conjugated secondary antibodies. Detection of UL131 and UL128, gels were blotted at 14V overnight onto FluoroTrans polyvinylidene fluoride membranes (Pall Corp., #bsp0161, 0.2 μm pore size) in transfer buffer consisting of 10 mM NaHCO₃, 3 mM Na₂CO₃,10% methanol (pH 9.9). Detection of non-reduced gH/gL complexes was performed. A custom rabbit antisera was raised against a synthetic peptide sequence matching UL148 positions 263-285 (Pacific Immunology, Ramona, Calif.). Rabbit antisera specific for UL130 and UL131, and mouse monoclonal anti-UL128 antibody clone 4810 were kindly provided by David C. Johnson (Oregon Health Sciences University, Portland, Oreg.) and Michael McVoy (Virginia Commonwealth University, Richmond, Va.). Rabbit antibodies to detect gO and gL.

Immunoprecipitation. Infected human foreskin fibroblasts (HFF) were lysed in RIPA buffer (50 mM Tris, pH 7.4, 150 mM NaCl, 1 mM EDTA, 1% Nonidet P-40, 0.1% SDS, 0.5% deoxycholate). Lysates were rotated at 4° C. overnight in the presence of rabbit anti-HA polyclonal antibody (Bethyl Laboratories, #A190-108A), mouse anti-gH monoclonal antibody 14-4b, or control IgG (as indicated), together with Protein A/G agarose beads (Thermo Scientific Pierce, #20423). Following washing steps, bound proteins were eluted by incubating for 5 min at 90° C. in SDS-PAGE sample buffer (20% glycerol, 4% SDS 100 mM Tris-Cl pH 6.8, 4 mM EDTA, 5% beta-mercaptoethanol).

qPCR. viral RNA levels were quantified using reverse-transcriptase quantitative PCR (RT-qPCR). Infected fibroblasts were harvested by trypsinization, and total RNA was extracted using an RNeasy Mini kit (Qiagen, Inc., Valencia, Calif.) including the optional on-column DNase digestion step. cDNA was generated by reverse transcription (RT) using the ProtoScript II First-Strand cDNA Synthesis Kit (New England BioLabs, Inc.). Following RT, samples were diluted 3-fold with water, and used as template for RT-qPCR. The ΔΔCT method was used to compare viral mRNAs levels, and RT-oPCR results for cellular GAPDH mRNA were used for normalization. Primers used to detect mRNAs for gH, gO, gL, UL128, UL130, and UL131 are shown in Table 1.

Oligonucleotides. Oligonucleotides were custom synthesized by Integrated DNA Technologies, Inc. (Coralville, Iowa).

The inventors' work has shown that blocking expression of the viral (glyco)protein encoded by the UL148 gene of human cytomegalovirus (HCMV) alters the cell tropism of the virus, increasing its tropism (or “infectivity”/potential to infect) for epithelial cells, and that these changes in cell tropism can be attributed to UL148 dependent effects on the expression of alternative forms of a viral glycoprotein complex referred to as glycoprotein H/glycoprotein L (gH/gL) which is present on HCMV virions (viral particles) and plays key roles in regulating the viral membrane fusion machinery necessary for HCMV to enter and infect a host cell. Specifically, glycoprotein O (gO), the product of the HCMV UL74 gene, participates in a three part (trimeric) complex with gH/gL, called “gH/gL/gO”, and the gH/gL/gO complex requires UL148 for its efficient maturation and incorporation into virions, particularly when UL128, UL130 and UL131 are also expressed by the virus. UL128 (also known as pUL128), UL130 (also known as pUL130) and UL131 (also referred to as UL131A or pUL131A) form a mutually exclusive five-part (pentameric) gH/gL complex called gH/gL/UL128-131 (or gH/gL/pUL128-131).

DISCUSSION and FURTHER EMBODIMENTS: Physicians in the neonatology, perinatology and pediatric fields refer to HCMV as one of the TORCH pathogens (“Toxoplasma”, “Other”, “Rubella”, “Cytomegalovirus”, “Herpes Simplex”) that are of special concern as a threat to the developing human fetus. U.S. Institute of Medicine has identified the development of an HCMV vaccine as a highest priority. The HCMV literature now has identified that maternal antibodies specific for the gH/gL/UL128-UL131 are particularly effective at blocking transmission of HCMV to the fetus. Because the inventors have identified UL148 to be a viral factor that influences the relative expression of gH/gL/gO to gH/gL/UL128-131, and because the gH/gL/UL128-131 complex is an important target for antibodies to protect against HCMV infection of the developing fetus, manipulation of the UL148 gene appears to represent a watershed in this technology as an approach to improve or optimize HCMV vaccines such that effective antibody responses are induced following immunization.

Furthermore, laboratories that study HCMV often encounter a problem in that when clinical isolates of HCMV are grown on human fibroblasts, which are the cells of choice for isolating and cultivating HCMV in the laboratory, the virus often accumulates mutations in the UL128, UL130 or UL131 that prevent expression of the gH/gL/UL128-131 complex. The inventors' work evidences that technologies to block expression of UL148 enable high-level replication of clinical HCMV strains on epithelial cell lines, such as ARPE-19. Because HCMV requires gH/gL/UL128-131 to enter epithelial cells but not fibroblasts, the ability to efficiently grow on epithelial cells clinical isolates of HCMV, or HCMVs that express both gH/gL/gO and gH/gL/UL128-131 is useful for maintaining the genetic stability of the UL128, UL130 and UL131 genes when cultivating virus for use in vaccines or other applications.

Examples of further embodiments to block or manipulate UL148 expression so as to alter the cell tropism of HCMV and/or to adjust the composition of virion gH/gL complexes include:

(i) Stable or transient expression in cells or cell lines of small interfering RNAs (e.g. short hairpin RNAs, microRNAs, siRNAs) directed against the UL148 mRNA of human cytomegalovirus, such that the mRNA encoding the UL148 polypeptide (protein) is either degraded and/or its translation is impaired.

(ii) Deletion, mutation, insertions or other alterations to the UL148 gene sequence in the context of any HCMV genome in a manner that would either prevent or reduce transcription of a UL148 messenger RNA (mRNA), such modifications may include modifications to UL148 protein coding sequences or modifications to upstream or downstream regulatory sequences that are involved in the transcription of the UL148 messenger RNA (mRNA) that would be translated into UL148 protein.

(iii) Deletion, mutation, insertions or other alterations to the UL148 gene sequence in the context of any HCMV genome in a manner that would either prevent translation of a functional UL148 protein or alter the function of the translated UL148 protein. Examples could include but are not limited to: the introduction of one or more stop (nonsense) codons in the UL148 gene, frameshift mutations in the UL148 gene, mutations in that cause one or more amino acid substitutions in the UL148 protein, mutations in the UL148 gene that introduce codons that are poorly used in human cells, mutations in the UL148 gene that introduce codons that would cause incorporation of a synthetic amino acid substitute, deletions in the UL148 gene that remove UL148 protein coding sequences or neighboring sequences involved in the production of a UL148 mRNA or in the translation into protein of the UL148 mRNA.

(iv) Genetic modifications of the UL148 gene such that the translated protein is fused to a “degron” peptide that either conditionally or constitutively destabilizes its expression.

(v) The use of small molecules or cell-permeable chemical compounds or agents for the purpose of blocking or altering the function of UL148 protein, for decreasing or destabilizing the expression of UL148 protein, for decreasing the transcription or stability of the UL148 messenger RNA (mRNA), or for otherwise causing the destruction or degradation of either UL148 mRNA message or the UL148 protein.

The gH/gL proteins are conserved and are widely assumed to play important roles in cell entry for all viruses in the herpesvirus family (HCMV is one of many different human herpesviruses, others include Varicella-Zoster virus, Herpes Simplex Virus 1 and 2, Epstein Barr Virus, Kaposi's sarcoma virus, and Human Herpesviruses 6A, 6B and 7). Several antibodies against HCMV gH/gL complex can completely block the ability of the virus to infect cells. But antibodies against gH/gL/UL128-131 are particularly potent (able to block infection at much lower concentrations than other gH/gL antibodies) at protecting against infection of epithelial cells, endothelial cells, and leukocytes and at blocking maternal transmission of the infection to the fetus. The requirement of gH/gL/UL128-131 for HCMV to enter these cell types can thus be seen as an “Achilles heel” of the virus as it must likely be able to enter these cell types to cross the placenta and infect/harm the fetus.

Additional embodiments demonstrating that ul148 plays regulating the composition of virion gh/gl complexes.

The inventors generated an HCMV virus that could not express UL148 by replacing a substantial portion of the UL148 allele in the viral genome with a drug selection marker, as shown above. The inventors additionally constructed mutant viruses in both HCMV strains TR and TB40/E in which the inventors replaced the UL148 gene in the viral genomes with a mutant version of the UL148 gene in which all of the seven in-frame methionine/start codons (present at amino acid positions 1, 64, 77, 200, 215, 298, and 299) were replaced with a nonsense or “stop” codon (e.g. TAG, TGA, TAA). This was done by en passant mutagenesis (references 1 and 2 below) in Escherichia coli of the cloned HCMV genomes, which has been previously cloned as infectious bacterial artificial chromosomes (BACs). Briefly, the inventors designed and executed a strategy for mutagenesis in which a version of the UL148 gene from HCMV strain TB40/E, starting 74 bp upstream of the first start codon of UL148 and 42 bp downstream of the native UL148 stop codon. TAG stop codons were incorporated in place of the native ATG methionine codons at UL148 amino acid positions 1, 64, 77, 200, 215, 298, and 299. This was custom synthesized by Integrated DNA Technologies, Inc. (Coralville, Iowa) in two parts and fused with 20 bp extensions to allow for assembly in a Gibson Assembly Reaction to an I-Sce-I homing endonuclease recognition site joined to a kanamycin resistance allele (ISce-I-Kan), and to a cloning plasmid pSP72 such that the I-Scel-Kan allele would be disrupting the mutant UL148 gene (“UL148 stop”), and such that the “UL148 stop” gene was disrupted by the kanamycin allele could be released by digestion of the resulting plasmid using the EcoRV restriction enzyme. The resulting plasmid was sequence verified. Other important features of the “UL148 stop” allele disrupted by the I-Scel Kan is that the I-Scel Kan cassette is flanked by a 47-bp direct repeated sequence of flanking UL148 stop sequence so that the I-Scel Kan cassette could be removed in via recombination between the repeats following induction in appropriate E. coli bacteria strain of I-Scel endonuclease and appropriate bacteriophage recombinase activities. To assure that the entire “UL148 stop” allele would be introduced into the HCMV BACs, the native UL148 allele was first replaced with a beta-lactamase gene (bla) from the pSP72 plasmid, also using en passant mutagenesis, leaving behind approximately 40 bp flanks matching the sequences at the edge of the UL148 stop cassette (released by EcoRV digestion of the pSP72 UL148stop ISce-I Kan plasmid) to enable it to be introduced in place of the bla selectable marker. By first replacing the UL148 gene with bla, the inventors were able to ensure all the engineered stop codon mutations would be included. Kanamycin antibiotic selection was used to select for E. coli colonies containing the UL148 stop cassette, referred to as “integrates” in en passant. Later I-Sce-I endonuclease activity was induced to induce a double stranded DNA break aside the I-Scel-Kan cassette, in tandem with heat shock to induce recombination enzymes that would catalyze recombination between the direct repeats flanking the I-Scel-Kan cassette, thereby removing it, and resulting in kanamycin sensitive “resolved” integrates. The resulting “resolves” E. coli colonies harboring UL148 stop mutant virus BACs were screened to identify colonies sensitive to both kanamycin and carbenicillin (a substrate of the bla gene product) antibiotics. BAC DNA was prepared from the UL148 stop colonies and transfected by electroporation into human foreskin fibroblast cells to reconstitute infectious virus, which was characterized (and compared to parental wild-type virus) by Western blot for expression of the gH/gL/gO complex and for expression of other viral glycoproteins.

En Passant mutagenesis references: 1.) Tischer B K, von Einem J, Kaufer B, Osterrieder N (2006) Two-step red-mediated re-combination for versatile high-efficiency markerless DNA manipulation in Escherichia coli. Biotechniques 40(2)191-197. 2.) Tischer B K, Smith G A, Osterrieder N (2010) En passant mutagenesis: A two-step markerless red recombination system. Methods Mol Biol 634:421-430.

OTHER ADDITIONAL DATA: To investigate the mechanism by which UL148 regulates alternative viral glycoprotein complexes in HCMV, the inventors generated new plasmids that express HA-epitope tagged UL148 and control proteins US11 and UL16 that also are type I transmembrane proteins that localize to the endoplasmic reticulum (ER), together plasmids expressing FLAG (and other) epitope-tagged versions of UL130, gO, gH and gL, and UL128, UL131. The experiments conducted show that UL148, when transfected together with UL130, is able to cause degradation of UL130, but that UL16 and US11 do not show this activity. Further shown was that co-expression of UL148 together with gH/gL is associated with degradation of gH/gL, and that gH/gL and UL148 co-immunoprecipitate reciprocally (when either is immunoprecipitated, the other can be detected), which suggests a protein-protein interaction. US11 and UL16 control proteins do not show any effect on gH/gL expression nor is reciprocal co-immunoprecipitation observed. These data further highlight the potential of UL148 to modulate expression of gH/gL complexes and suggest that UL148 may favor gH/gL/gO expression in part by binding and causing retention of UL130 and or UL130 containing complexes in the endoplasmic reticulum (ER), and/or that UL148 may act within the ER to degrade UL130 containing complexes.

While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/We claim:

1. A method of changing a tropism of a herpes virus comprising the steps of:
one of deleting, substituting, or modifying a UL148 gene and interfering with or modifying an expression of the UL148 gene.

2. The method of claim 1, wherein the herpes virus is human cytomegalovirus.

3. The method of claim 1, wherein the change in tropism enhances epithelial cell tropism.

4. The method of claim 3 wherein a epithelial cell line is ARPE-19.

5. A method of increasing a ratio of gH/gL/UL128-131 to gH/gL/gO in a herpes virus comprising the steps of:
one of deleting, substituting, or modifying a UL148 gene and interfering with or modifying an expression of the UL148 gene.

6. The method of claim 5, wherein the herpes virus is human cytomegalovirus.

7. A method of preparing a vaccine for immunization against a herpes virus comprising the steps of:
one of deleting, substituting, or modifying a UL148 gene and interfering with or modifying an expression of the UL148 gene.

8. The method of claim 7, wherein the herpes virus is human cytomegalovirus.

9. The method of claim 7, further comprising the step of using a manmade organism TB_148^{HA}.

10. The method of claim 7, further comprising the step of using a manmade organism TB_Δ148.

11. The method of claim 7, further comprising the step of using a manmade organism ADr131_UL148^{HA}.

12. The method of claim 7, further comprising the steps of:
obtaining a solution containing either herpes viruses or an infectious herpesvirus genome cloned in Escherichia coli as a bacterial artificial chromosome (BAC);
deleting, substituting, or modifying the UL148 gene of the human cytomegalovirus or of a functionally analogous gene of any beta or gamma herpes virus in which alternative forms of gH/gL complexes are found on virions, or interfering with or modifying an expression of the UL148 gene of the herpes virus;
using permissive cells to cultivate the herpes virus and/or to reconstitute an infectious virus from BAC DNA;
micro-filtering a herpes virus solution to remove blood cells and other larger particles or impurities while letting the herpes virus pass through; and
diluting a filtrate containing the herpes virus with a sterile saline solution, thereby forming the vaccine.

13. The method of claim 12 further comprising the step of killing, attenuating, or otherwise inactivating the herpes virus.

14. The method of claim 7 further comprising the step of producing one of stable and transient expression in cells or cell lines of interfering RNAs directed against UL148 mRNA of human cytomegalovirus, such that mRNA encoding a UL148 polypeptide is one of degraded and impaired in translation.

15. The method of claim 7 further comprising the step of preforming one of a deletion, mutation, insertion and other alteration to a UL148 gene sequence which one of prevents and reduces transcription of a UL148 messenger RNA (mRNA).

16. The method of claim 15 further comprising the step of modifying a UL148 protein coding sequence and modifying an upstream or downstream regulatory sequence that is involved in a transcription of the UL148 messenger RNA that would be translated into a UL148 protein.

17. The method of claim 7 further comprising the step of performing one of a deletion, mutation, insertion and other alteration to a UL148 gene sequence which one of prevents translation of a functional UL148 protein and alters a function of a translated UL148 protein.

18. The method of claim 17 further comprising the step of introducing one of stop (nonsense) codons in the UL148 gene, frameshift mutations in the UL148 gene, mutations in that cause one or more amino acid substitutions in the UL148 protein, mutations in the UL148 gene that introduce codons that are poorly used in human cells, mutations in the UL148 gene that introduce codons that would cause incorporation of a synthetic amino acid substitute, deletions in the UL148 gene that remove one of UL148 protein coding sequences and neighboring sequences involved in one of a production of a UL148 mRNA and in a translation into protein of the UL148 mRNA.

19. The method of claim 7 further comprising the step of producing genetic modifications of the UL148 gene such that a translated protein is fused to a degron peptide that either conditionally or constitutively destabilizes the translated protein expression.

20. The method of claim 7 further comprising the steps of
blocking of altering a function of a UL148 protein with small molecules or cell-permeable chemical compounds or agents,
one of decreasing and destabilizing the expression of UL148 protein, and
one of decreasing a transcription or stability of a UL148 messenger RNA (mRNA), and causing a destruction or degradation of either a UL148 mRNA message or the UL148 protein.
